(19) United States
(12) Patent Application Publication
(10) Pub. No.: US 2015/0305826 A1
(43) Pub. Date: Oct. 29, 2015

(54) METHOD FOR PRODUCING RADIOPAQUE MEDICAL IMPLANTS

(71) Applicants: STRYKER CORPORATION, Kalamazoo, MI (US); STRYKER EUROPEAN HOLDINGS I, LLC, Kalamazoo, MI (US)

(72) Inventors: Siddharth Loganathan, Santa Clara, CA (US); Brent C. Gerberding, San Jose, CA (US)

(21) Appl. No.: 14/697,029

(22) Filed: Apr. 27, 2015

Related U.S. Application Data
(60) Provisional application No. 61/985,621, filed on Apr. 29, 2014.

(51) Int. Cl. A61B 19/00; A61F 2/852; A61F 2/94

(52) U.S. Cl. CPC A61B 19/54; A61F 2/94; A61F 2/852; A61B 2019/5466; A61F 2250/0067

(57) ABSTRACT

An implantable medical device includes a structural element having a core layer or region of radiolucent material; a layer of radiopaque material overlaying the core layer or region of radiolucent material; and an outer layer of radiolucent material overlaying the layer of radiopaque material, the outer layer of radiolucent material having a same or greater hardness as the layer of radiopaque material.

### RELATED APPLICATION DATA

The present application claims the benefit under 35 U.S.C. §119 to U.S. provisional patent application Ser. No. 61/985,621, filed Apr. 29, 2014. The foregoing application is hereby incorporated by reference into the present application in its entirety.

### FIELD OF THE INVENTION

The inventions disclosed herein are in the field of medical implants, such as stents, and more particularly pertain to implants having at least one radiopaque layer encased by an outer protective layer, and methods for making such implants.

### BACKGROUND

Implantable medical devices such as arterial stents used to maintain vessel patency and/or divert blood flow are well-known. Such devices may be made from a single piece of material (e.g., a laser-cut hypo tube), or from multiple elements, such as a woven mesh formed from numerous individual strands of material. Other constructions are also possible. Reference is made to U.S. patent application Ser. No. 14/139,815, entitled “Multi-layer Stent,” the full disclosure of which is incorporated herein by reference.

The degree of radiodensity (or “radiopacity”) of a material refers to the relative inability of electromagnetic radiation, particularly x-rays, to pass through the material. Materials that inhibit the passage of electromagnetic radiation are referred to as being “radiodense” or “radiopaque”. The term radiopaque refers to the relatively opaque whitish appearance of radiodense materials or substances on radiographic (e.g., fluoroscopic) imaging displays, compared with the relatively darker appearance of less dense materials. Conversely, the “radiolucency” of a material refers to the relative transparency (or “transradiancy”) of the material to x-rays, with materials that allow radiation to pass more freely being referred to as “radiolucent”. It will be appreciated that the radiopacity of a material, and conversely, the radiolucency of a material, is in each instance a relative characteristic and matter of degree. Materials that are considered to be radiolucent may still be vaguely detectable, and conversely, materials that are considered to be radiopaque be still require some enhancement for ease in visualization. Nonetheless, materials that can be readily observed or detected on a fluoroscopic x-ray display are referred to herein as radiopaque materials, and materials that cannot be readily observed or detected on a fluoroscopic x-ray display are referred to herein as radiolucent materials.

Medical devices that are implanted in the vascular system (such as a stent) are typically highly flexible and resilient. Such devices are formed of materials selected for superior in-vivo performance, such as stainless steel or a nickel titanium alloy. However such materials are typically radiolucent, thus an additional, radiopaque material (a “radiopacifier”) must be added to the device to enhance visualization during an implantation procedure to ensure the device is implanted at the desired location in the vasculature. For example, a radiopaque material may be in the form of a coating that is applied to the surface of an implant. But, the durability of such coatings is a challenge once the layer is exposed to in-vivo conditions, due to fretting and/or abrasion when implants deployed in an overlapping arrangement.

Applying a coating to only the outer surface of an implant leads to the problem of fretting because the “sides” of the layers of radiopaque coating and implant core material are exposed on the surfaces of the implant perpendicular to the outer surface. The exposed “sides” of the layers of coating an implant core material can “catch” during delivery or other interactions with different surfaces and cause the layers to delaminate.

For example, FIGS. 1A-1C depict a structural element (e.g., a strut) of a prior art stent 10, which includes a core layer or region 12 (e.g., made from Nitinol™) with an outer surface 14 covered by a radiopaque layer 16. The radiopaque layer 16 can be made from a biocompatible radiopaque metal such as gold, platinum, iridium, palladium, and rhodium. As shown in FIG. 1A, the “sides” 18, 20 of the core 12 and the layer 16 are exposed. If those “sides” 18, 20 are disposed at the leading/distal edge of the stent 10, distal sliding of the stent 10 during delivery will exert frictional forces on the leading edge “side” 20 of the layer 16, urging the layer 16 to delaminate from the core 12. If those “sides” 18, 20 are disposed perpendicular or diagonal to the leading edge of the stent 10, radial expansion of the stent 10 will exert similar frictional forces on the “side” 20 of the layer 16. If those “sides” 18, 20 are disposed at the trailing/proximal edge of the stent 10, proximal sliding of the stent 10, for instance during re-sheathing of a partially delivered stent, will exert similar frictional forces on the “side” 20 of the layer 16.

FIGS. 2A-2C depict a structural element (e.g., a strut) of another prior art stent 10, which is similar to the structural element depicted in FIGS. 1A-1C, except for the addition of an outer Nitinol™ layer 22 configured to protect the radiopaque layer 16. The outer Nitinol™ layer 22 protects the radiopaque layer 16, but exacerbates the fretting problem by adding another layer interface and exposed “side” 24 (of the outer Nitinol™ layer 22) that is susceptible to delamination.

### SUMMARY

The disclosed inventions are directed to implantable medical devices having one or more structural elements made of at least one radiopaque material layer with an overlying outer protective layer, and to methods for making such implantable devices.

In various disclosed embodiments, an implantable medical device comprises a structural element made of a core layer or region of radiolucent material, a layer of radiopaque material overlaying the core layer or region of radiolucent material, and an outer layer of radiolucent material overlaying the layer of radiopaque material, the outer layer of radiolucent material having a same or greater hardness as the layer of radiopaque material. In some embodiments, the layer of radiopaque material is disposed along a longitudinal axis of the structural element. In other embodiments, the layer of radiopaque material is disposed transverse to a longitudinal axis of the structural element. The layer of radiopaque material may substantially encases the core layer or region of radiolucent material, and wherein the outer layer of radiolucent material substantially encases the layer of radiopaque material. One or both of the outer layer of radiolucent material and the core layer or region of radiolucent material may be made from a metal or metal alloy. By way of example, the outer layer of radiolucent material may be made from a nickel-titanium alloy that is applied in a super elastic state to the device.

In one embodiment, the structural element made be made out of a core layer or region of radiolucent material, a first layer of radiopaque material overlaying the core layer or region of radiolucent material, an inner layer of radiolucent material at least partially overlaying the first layer of radiopaque material, a second layer of radiopaque material at least partially overlaying the inner layer of radiolucent material; and an outer layer of radiolucent material at least partially overlaying the second layer of radiopaque material, having a same or greater hardness as the second layer of radiopaque material. For example, the first layer of radiopaque material may substantially encase the core layer or region of radiolucent material, wherein the inner layer of radiolucent material substantially encases the first layer of radiopaque material, the second layer of radiopaque material substantially encases the inner layer of radiolucent material, and the outer layer of radiolucent material substantially encases the second layer of radiopaque material. One or both of the inner layer of radiolucent material and outer layer of radiolucent material comprises a metal or metal alloy. For example, the outer layer of radiolucent material may be formed from a nickel-titanium alloy that is applied in a superelastic state to the device.

In various further disclosed embodiments, an implantable medical device comprises structural element constructed out of a core region or layer of radiolucent material, a plurality of layers of radiopaque material overlaying the core region or layer of radiolucent material, and a plurality of outer layers of radiolucent material, with respective layers of radiolucent material layers overlaying respective layers of radiopaque material, the respective layers of radiolucent material having a same or greater hardness as the respective layers of radiopaque material. Without limitation, the respective layers of radiopaque material and radiolucent material may be disposed along a longitudinal axis of the structural element. In one embodiment, the respective layers of radiopaque material and radiolucent material defining a plurality of grooves disposed along the longitudinal axis of the structural element, the device further comprising a therapeutic agent carried in one or more of the grooves. Alternatively, the respective layers of radiopaque material and radiolucent material may be disposed transverse to a longitudinal axis of the structural element. In one such embodiment, the respective layers of radiopaque material and radiolucent material defining a plurality of grooves disposed along the longitudinal axis of the structural element, the device further comprising a therapeutic agent carried in one or more of the grooves.

These and other aspects of the disclosed inventions will be apparent from the following detailed description, and the appended claims, particularly when considered in conjunction with the accompanying drawings in which like parts bear like reference numerals.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings that depict embodiments of the disclosed inventions depict only typical embodiments and are not therefore to be considered limiting of its scope.

FIG. 1A is a detailed perspective view of a structural element of a prior art stent.

FIGS. 1B and 1C are detailed side views of the structural element depicted in FIG. 1A.

FIG. 2A is a detailed perspective view of a structural element of a prior art stent.

FIGS. 2B and 2C are detailed side views of the structural element depicted in FIG. 2A.

FIGS. 3A and 3B are detailed perspective and longitudinal cross-sectional views of a structural element of a stent according to one disclosed embodiment.

FIG. 4 is a cross-sectional view of a structural element of a stent according to another disclosed embodiment.

FIG. 5 is a fluoroscopic image of a stent according to one disclosed embodiment and a prior art stent.

FIG. 6 is a flow chart depicting a method of manufacturing a stent according to a disclosed embodiment.

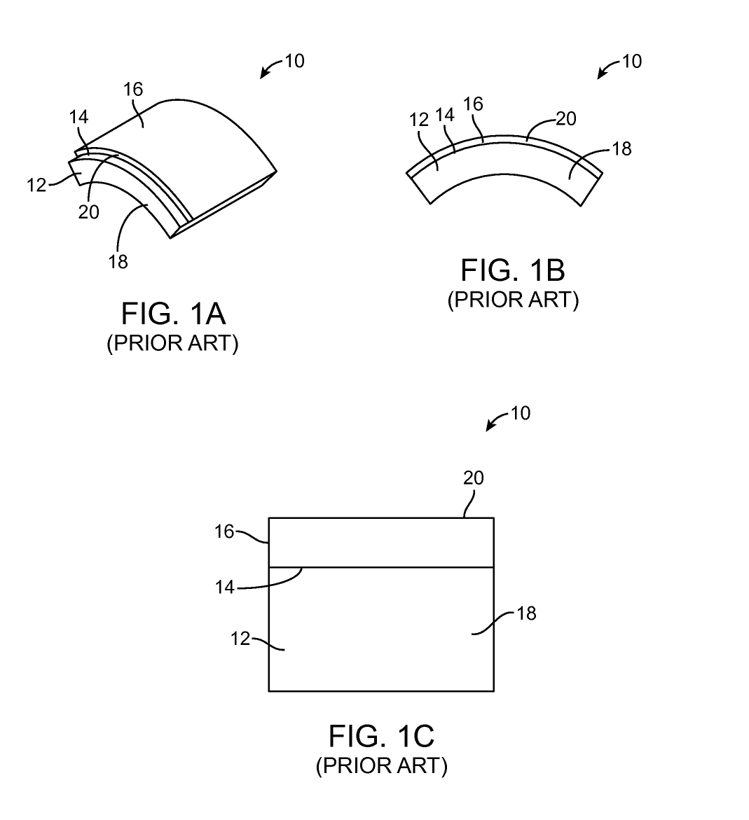

### DETAILED DESCRIPTION

Various embodiments of the disclosed inventions are described hereinafter with reference to the figures. The figures are not necessarily drawn to scale, the relative scale of select elements may have been exaggerated for clarity, and elements of similar structures or functions are represented by like reference numerals throughout the figures. It should also be understood that the figures are only intended to facilitate the description of the embodiments, and are not intended as an exhaustive description of, or as a limitation on the scope of, the disclosed inventions, which are defined only by the appended claims and their equivalents. In addition, an illustrated embodiment of the disclosed inventions needs not have all the aspects or advantages shown, and an aspect or an advantage described in conjunction with a particular embodiment of the disclosed inventions is not necessarily limited to that embodiment and can be practiced in any other embodiments even if not so illustrated.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. All numeric values are herein assumed to be modified by the term “about,” whether or not explicitly indicated. The terms “about” and “approximately” generally refer to a range of numbers that one of skill in the art would consider equivalent to the recited value (i.e., having the same function or result). In many instances, the terms “about” and “approximately” may include numbers that are rounded to the nearest significant figure. As used in this specification and the appended claims, numerical ranges include both endpoints and all numbers included within the range. For example, a range of 1 to 5 inches includes, without limitation, 1, 1.5, 2, 2.75, 3, 3.80, 4 and 5 inches.

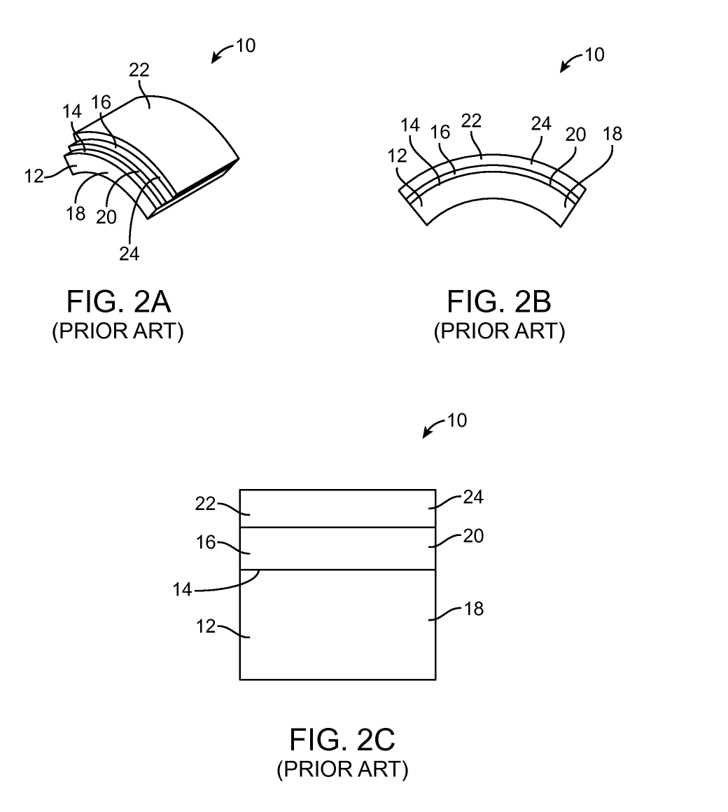

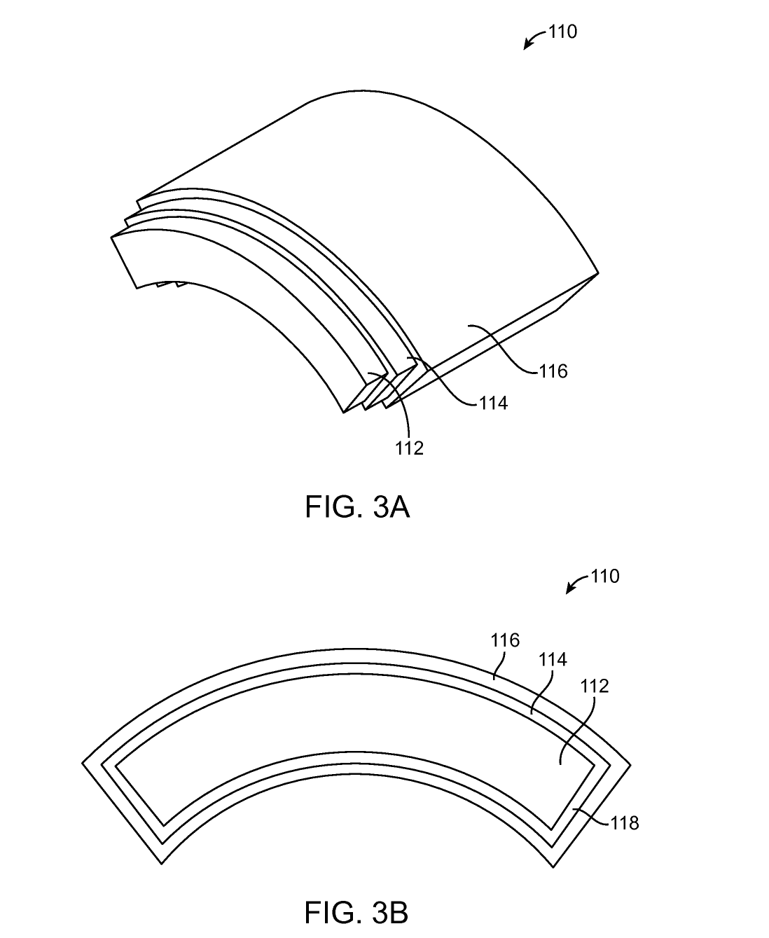

Turning back to the drawings, FIG. 3 illustrates a structural element (e.g., a strut) of an implant (e.g., a stent) 110 according to one disclosed embodiment, which includes a core layer or region 112, a radiopaque layer 114 and an outer protective layer 116. The core layer or region 112 can be made of a metal or a metal alloy, such as stainless steel or Nitinol™. The radiopaque layer 114 can be made from a biocompatible radiopaque metal such as gold, platinum, iridium, palladium, and rhodium. The outer protective layer 116 can be made from Nitinol™. Unlike the prior art stent 10 depicted in FIGS. 2A-2C, the radiopaque layer 114 substantially encases the core layer or region 112 and the outer protective layer 116 substantially encases the radiopaque layer 114. The outer protective layer 116 has a hardness equal to or greater than the hardness of the radiopaque layer 114.

Because the radiopaque and outer protective layers 114, 116 substantially encase the respective core and radiopaque layers 112, 114, the layers of the structural element do not have any “open sides.” The structure greatly reduces delamination of the layers resulting from frictional forces during deployment of the stent 110. In addition to reducing the fretting problem, the structure also reduces galvanic effect on the radiopaque layer 114 and problems with delamination of the core and outer protective layers 112, 116. Further, the outer protective layer 116 is more damage tolerance and has a lower coefficient of friction than the polymeric coatings (e.g., parylene) on current stents.

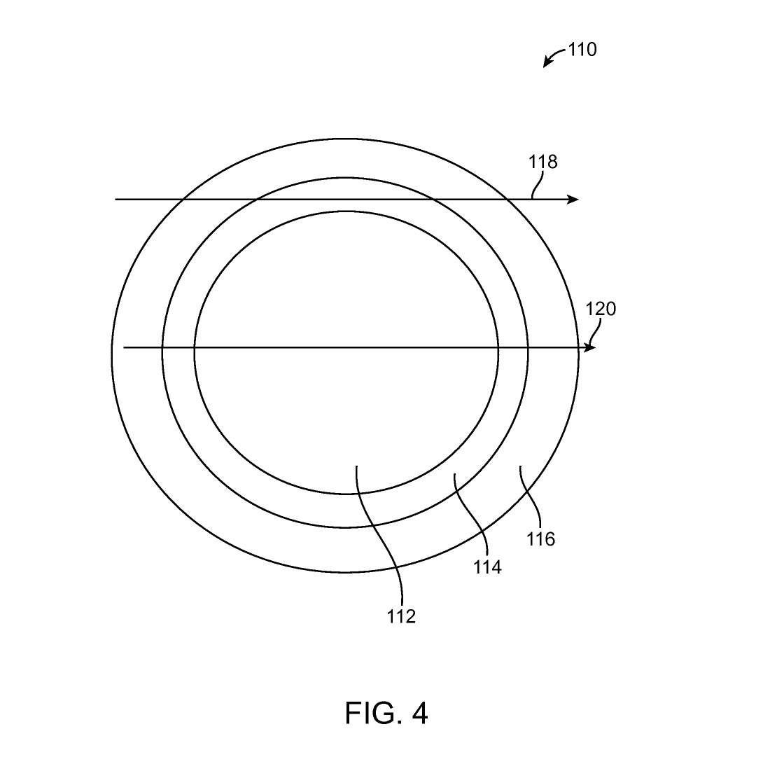

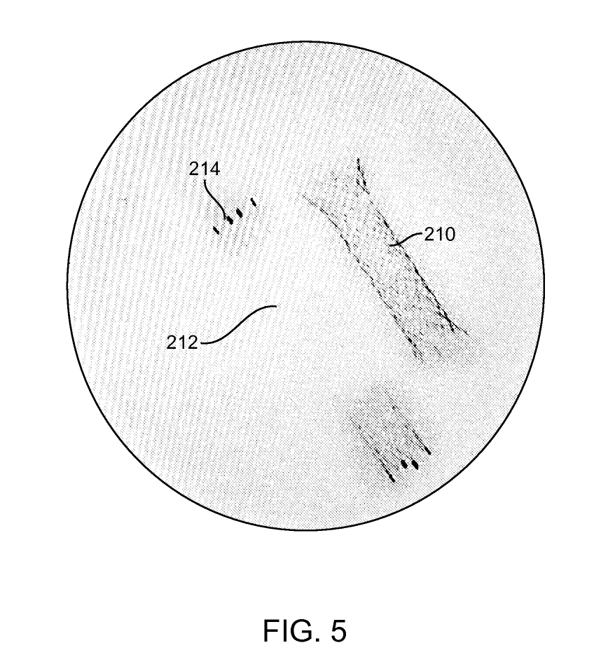

The structure also increases radiopacity because of the greater mass of radiopaque material at the edges of structural elements than through the centers of those elements. As shown in FIG. 4, even in a structural element having a circular cross-section, the side 118 of the structural element includes a greater mass of radiopaque material in the radiopaque layer 114 than the center 120 of the structural element. As a result, imaging signals (e.g., X-rays) passing through the sides 118 of structural elements according to various disclosed embodiments are absorbed to a greater extent, leading to greater contrast in the image, as shown in FIG. 5. FIG. 5 depicts an exemplary fluoroscopic image of a first stent 210 with a substantially encasing radiopaque coating and a second stent 212 without a substantially encasing radiopaque coating. As shown in FIG. 5, only the markers 214 on the second stent 212 (without a substantially encasing radiopaque coating) are visible under fluoroscopy, while the entire first stent 212 is visible under fluoroscopy. This effect is increased in structural elements having more linear sides, as shown in FIG. 3B.

In some embodiments, the outer protective layer 116 is made from a shape memory metal (e.g., Nitinol™), which is added to the structural member in its superelastic state while the stent is in its expanded condition. In the superelastic state, a shape memory metal has an elasticity 10 to 30 times that of ordinary metal. Accordingly, the outer protective layer 116 in such embodiments contributes to the chronic outward force of the stent 110.

In some embodiments, two or more radiopaque layers 114 can be added to the structural member with a corresponding number of protective layers 116 separating the radiopaque layers 114. In some embodiments the radiopaque material forming the radiopaque layer 116 can be applied/added to the structural member as a series of bands (instead of a continuous shell around the core layer 112). These bands can be parallel to the longitudinal axis of the structural element, transverse to the longitudinal axis, or any angle in between relative to the longitudinal axis.

In some embodiments, the metallic material forming the outer protective layer 116 can be applied/added to the structural member as a series of bands (instead of a continuous shell around the radiopaque layer 114). These bands can be parallel to the longitudinal axis of the structural element, transverse to the longitudinal axis, or any angle in between relative to the longitudinal axis. In some embodiments, both the radiopaque material and the metallic material can be applied/added to the structural member as a series of bands. The bands of radiopaque material and metallic material can be parallel to, perpendicular to, or at various angles with respect to each other. In all of the embodiments with either bands of radiopaque material and/or bands of metallic material, the bands form grooves and cavities, which can be filled with anti-thrombotic, restenosis inhibiting, and/or other therapeutic agents.

In another embodiment, grooves formed by the bands of radiopaque and/or metallic material can be used to mechanically align and couple another device (e.g., another stent) to the stent 110. For instance, grooves on an outer surface of a first stent 110 can be align with grooves on an inner surface of a second stent 110 to couple the second stent 110 on top of the first stent 110. In yet another embodiment, the “bands” can form an approximately helical groove into which a radiopaque wire/ribbon can be aligned in a spiral and secured. In still another embodiment, the “bands” can form grooves that align radiopaque wires/ribbons to facilitate weaving of the wires/ribbons. While the grooves in these embodiments are described as being defined by “bands,” the radiopaque and/or metallic material can form discontinuous structures that define grooves as long as the discontinuous structures provide sufficient mechanical interference to align and couple as described above.

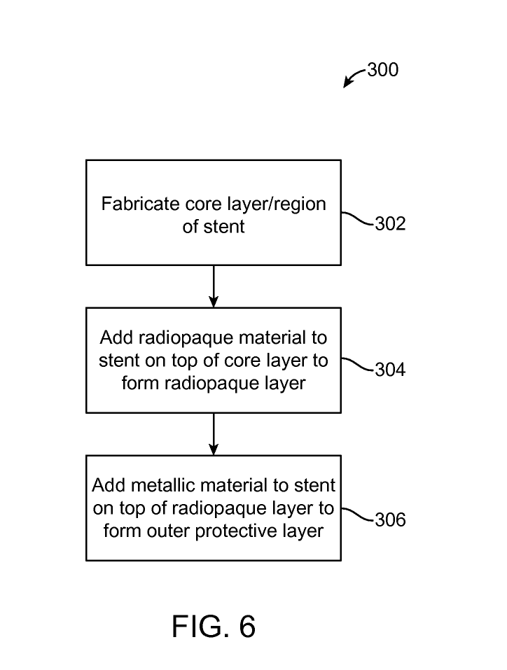

FIG. 6 depicts a method 300 of manufacturing a stent according to a disclosed embodiment. At step 302, the core layer or region 112 of the stent 110 is fabricated through processes such as laser cutting. At step 304, radiopaque material is added to the stent 110, on top of the core layer 112, through processes such as sputtering, vapor deposition, ion beam deposition, and cathodic arc deposition, forming the radiopaque layer 114. The parameters for these coating processes are adjusted so that the radiopaque material is added to all surfaces of the structural elements of the stent 110. In some embodiments, the core layer 112 is completely encased in the radiopaque material forming the radiopaque layer 114. At step 306, metallic material (e.g., Nitinol™) is added to the growing stent 110, on top of the radiopaque layer 114, through the processes described in step 304, forming the outer protective layer 116.

Steps 304 and 306 can be repeated to form a stent 110 with a plurality of radiopaque layers 114 separated by respective protective layers 116. In embodiments where radiopaque material and/or metallic material are added/applied as bands, a filler/masking material can be applied to the layer supporting the radiopaque and/or metallic material before application of the radiopaque and/or metallic material to form the bands. After the bands are formed, the filler/masking material can be removed through a thermal or chemical process.

It will be appreciated that elements or components shown with any embodiment herein are exemplary for the specific embodiment and may be used on or in combination with other embodiments disclosed herein. While the disclosed and described embodiments are susceptible to various modifications, and alternative forms, specific examples thereof have been shown in the drawings and are herein described in detail. It should be understood, however, that the disclosed inventions are not to be limited to the particular forms or methods disclosed, but to the contrary, cover all modifications, equivalents and alternatives falling within the scope of the appended claims.

What is claimed is:

1. An implantable medical device comprising a structural element, the structural element comprising:
a core layer or region of radiolucent material;
a layer of radiopaque material overlaying the core layer or region of radiolucent material; and
an outer layer of radiolucent material overlaying the layer of radiopaque material, the outer layer of radiolucent material having a same or greater hardness as the layer of radiopaque material.

2. The implantable device of claim 1, wherein the layer of radiopaque material substantially encases the core layer or region of radiolucent material, and wherein the outer layer of radiolucent material substantially encases the layer of radiopaque material.

3. The implantable device of claim 1, wherein one or both of the outer layer of radiolucent material and the core layer or region of radiolucent material comprises a metal or metal alloy.

4. The implantable device of claim 3, wherein the outer layer of radiolucent material comprises a nickel-titanium alloy that is applied in a superelastic state to the device.

5. The implantable medical device of claim 1, wherein the layer of radiopaque material is disposed along a longitudinal axis of the structural element.

6. The implantable medical device of claim 1, wherein the layer of radiopaque material is disposed transverse to a longitudinal axis of the structural element.

7. An implantable medical device comprising structural element, the structural element comprising:
a core layer or region of radiolucent material;
a first layer of radiopaque material overlaying the core layer or region of radiolucent material;
an inner layer of radiolucent material at least partially overlaying the first layer of radiopaque material;
a second layer of radiopaque material at least partially overlaying the inner layer of radiolucent material; and
an outer layer of radiolucent material at least partially overlaying the second layer of radiopaque material, having a same or greater hardness as the second layer of radiopaque material.

8. The implantable device of claim 7, wherein the first layer of radiopaque material substantially encases the core layer or region of radiolucent material.

9. The implantable device of claim 8, wherein the inner layer of radiolucent material substantially encases the first layer of radiopaque material.

10. The implantable device of claim 9, wherein the second layer of radiopaque material substantially encases the inner layer of radiolucent material.

11. The implantable device of claim 10, wherein the outer layer of radiolucent material substantially encases the second layer of radiopaque material.

12. The implantable device of claim 7, wherein one or both of the inner layer of radiolucent material and outer layer of radiolucent material comprises a metal or metal alloy.

13. The implantable device of claim 12, wherein the outer layer of radiolucent material comprises a nickel-titanium alloy that is applied in a superelastic state to the device.

14. The implantable medical device of claim 7, wherein the first and second layers of radiopaque material are disposed along a longitudinal axis of the structural element.

15. The implantable medical device of claim 7, wherein the first and second layers of radiopaque material are disposed transverse to a longitudinal axis of the structural element.

16. An implantable medical device comprising structural element, the structural element comprising:
a core region or layer of radiolucent material;
a plurality of layers of radiopaque material overlaying the core region or layer of radiolucent material; and
a plurality of outer layers of radiolucent material, with respective layers of radiolucent material layers overlaying respective layers of radiopaque material, the respective layers of radiolucent material having a same or greater hardness as the respective layers of radiopaque material.

17. The implantable medical device of claim 16, wherein the respective layers of radiopaque material and radiolucent material are disposed along a longitudinal axis of the structural element.

18. The implantable medical device of claim 17, the respective layers of radiopaque material and radiolucent material defining a plurality of grooves disposed along the longitudinal axis of the structural element, the device further comprising a therapeutic agent carried in one or more of the grooves.

19. The implantable medical device of claim 16, wherein the respective layers of radiopaque material and radiolucent material are disposed transverse to a longitudinal axis of the structural element.

20. The implantable medical device of claim 19, the respective layers of radiopaque material and radiolucent material defining a plurality of grooves disposed along the longitudinal axis of the structural element, the device further comprising a therapeutic agent carried in one or more of the grooves.
